(19) United States
(12) Patent Application Publication
(10) Pub. No.: US 2025/0319251 A1
(43) Pub. Date: Oct. 16, 2025

(54) Drug Delivery Device With Movement Sensing

(71) Applicant: Becton, Dickinson and Company, Franklin Lakes, NJ (US)

(72) Inventors: Steve Beguin, Nenzlingen (CH); Wendy D. Woodley, Cary, NC (US); Sylvine Raverdy-Wilson, River Edge, NJ (US); Natasha G. Bolick, Raleigh, NC (US); Christian Sandmann, Wayne, NJ (US); Stuart Plascott, Foxrock (IE)

(21) Appl. No.: 18/866,573

(22) PCT Filed: Jun. 6, 2023
(86) PCT No.: PCT/US2023/024545

§ 371 Date: Nov. 18, 2024

Related U.S. Application Data
(60) Provisional application No. 63/349,797, filed on Jun. 7, 2022.

(51) Int. Cl. A61M 5/172; A61M 5/142; G16H 20/17

(52) U.S. Cl. CPC A61M 5/1723; A61M 5/14248; G16H 20/17; A61M 2205/13; A61M 2205/18

(57) ABSTRACT

A drug delivery device that includes a sensor to evaluate movement of the device relative to the body it is attached to and at least one processor in communication with the sensor, where the at least one processor is programmed or configured to receive data associated with the sensor from the sensor, determine a characteristic associated with the sensor based on the data received from the sensor, determine whether the characteristic associated with the sensor satisfies a threshold, and perform an action based on determining that the data received from the sensor satisfies the threshold.

### CROSS-REFERENCE TO RELATED APPLICATION

The present application claims priority to U.S. Provisional Application No. 63/349,797, entitled “Drug Delivery Device with Movement Sensing”, filed Jun. 7, 2022, the entire disclosure of which is hereby incorporated by reference in its entirety.

### BACKGROUND

Medical injectors (e.g., wearable drug delivery systems, wearable injection devices, wearable automatic injectors, on-body injectors (OBIS), on-body delivery systems (OBDS), autoinjectors, IV catheters, IV ports, transdermal delivery patches, etc.) may provide drug therapy to a patient when the patient is at a location that is remote from a clinical facility and/or while being worn discretely under the patient's clothing. A wearable injector can be applied to the patient's skin and may be configured to automatically or based on a user's command, deliver a dose of a drug (e.g., a pharmaceutical composition, a medication, etc.) when the wearable injector is applied to (e.g., positioned on) the patient's skin, such as after a delay of a predetermined time period or immediately upon application of the wearable injector. In some instances, the wearable injector may deliver the drug to the patient for a duration of time. After the wearable injector delivers the pharmaceutical composition to the patient, the patient may subsequently remove and dispose of the device.

In some situations, when a wearable injector is applied to a body of the patient, the wearable injector may move. For example, after the wearable injector is applied to a limb of the patient, the position of the wearable injector may change based on movements of the limb of the patient. In some instances, the wearable injector and components thereof, such as a cannula and/or a needle, may be displaced from an intended position based on movement of the patient. Movement of the wearable injector can disrupt proper introduction of a drug to the patient. In some instances, if extensive movement occurs, the wearable injector may come out of the patient, interrupting delivery of the drug.

Accordingly, it is beneficial to determine whether a wearable injector is properly positioned on a body of a patient to ensure correct delivery of a drug to the patient.

### SUMMARY

Accordingly, provided are improved systems, devices, products, apparatus, and/or methods for sensing movement of a drug delivery device.

Further non-limiting embodiments or aspects are set forth in the following numbered clauses:

Clause 1. A drug delivery device, for delivering a drug to a patient, including a cannula in fluid communication with a reservoir, at least one sensor, and at least one processor in communication with the sensor. The at least one processor is programmed or configured to receive data associated with the sensor from the sensor, determine a characteristic associated with the sensor based on the data received from the sensor, wherein the characteristic is relative movement between the drug delivery device and the patient, determine whether the characteristic associated with the sensor satisfies a threshold, and perform an action based on determining that the data received from the sensor satisfies the threshold.

Clause 2. A drug delivery device of clause 1, wherein the sensor is an acceleration sensor, wherein, when receiving the data from the sensor, the at least one processor is programmed or configured to receive data associated with acceleration of the acceleration sensor from the acceleration sensor, wherein, when determining the characteristic associated with the sensor, the at least one processor is programmed or configured to determine a velocity of the acceleration sensor based on the data associated with acceleration of the acceleration sensor. When determining whether the characteristic associated with the sensor satisfies the threshold, the at least one processor is programmed or configured to determine whether the velocity of the acceleration sensor satisfies a threshold value of velocity, and wherein, when performing the action, the at least one processor is programmed or configured to perform the action based on determining that the velocity of the acceleration sensor satisfies the threshold value of velocity.

Clause 3. A drug delivery device of clause 1, wherein the sensor is an acceleration sensor, wherein, when receiving the data from the sensor, the at least one processor is programmed or configured to receive data associated with acceleration of the acceleration sensor from the acceleration sensor. When determining the characteristic associated with the sensor, the at least one processor is programmed or configured to determine an amount of inertial force of acceleration sensor based on the data associated with acceleration of the acceleration sensor. When determining whether the characteristic associated with the sensor satisfies the threshold, the at least one processor is programmed or configured to determine whether the amount of inertial force of the acceleration sensor satisfies a threshold value of force, and wherein, when performing the action, the at least one processor is programmed or configured to perform the action based on determining that the inertial force of the acceleration sensor satisfies the threshold value of force.

Clause 4. A drug delivery device of clause 3, wherein, when determining the amount of inertial force, the at least one processor is programmed or configured to determine a first amount of inertial force in a first direction based on first data associated with acceleration of the acceleration sensor in a first direction, determine a second amount of inertial force in a second direction based on second data associated with acceleration of the acceleration sensor in a second direction. When determining whether the inertial force of the acceleration sensor satisfies the threshold value of force, the at least one processor is programmed or configured to determine whether the first amount of inertial force in the first direction satisfies a first threshold value of force, determine whether the second amount of inertial force in the second direction satisfies a second threshold value of force, and when performing the action, the at least one processor is programmed or configured to perform the action based on determining that the first amount of inertial force in the first direction satisfies the first threshold value of force or that the second amount of inertial force in the second direction satisfies the second threshold value of force.

Clause 5. A drug delivery device of clause 1, wherein the sensor is an acceleration sensor, wherein, when receiving the data from the sensor, the at least one processor is programmed or configured to receive data associated with acceleration of the acceleration sensor from the acceleration sensor. When determining the characteristic associated with the sensor, the at least one processor is programmed or configured to determine a first vector value of acceleration for the acceleration sensor in a first direction based on the data associated with acceleration of the acceleration sensor, determine a second vector value of acceleration for the acceleration sensor in a second direction based on the data associated with acceleration of the acceleration sensor, and determine a resultant acceleration vector of acceleration for the acceleration sensor based on the first vector value of acceleration of the acceleration sensor in the first direction and the second vector value of acceleration of the acceleration sensor in the second direction. When determining whether the characteristic associated with the sensor satisfies the threshold, the at least one processor is programmed or configured to determine whether the resultant acceleration vector of acceleration for the acceleration sensor satisfies a threshold value of acceleration. When performing the action, the at least one processor is programmed or configured to perform the action based on determining that the resultant acceleration vector of acceleration for the acceleration sensor satisfies the threshold value of acceleration.

Clause 6. A drug delivery device of clause 1, wherein the sensor is an acceleration sensor, wherein the at least one processor is further programmed or configured to determine an orientation of the acceleration sensor. When determining the characteristic associated with the sensor, the at least one processor is programmed or configured to determine the characteristic associated with the acceleration sensor based on the orientation of the acceleration sensor.

Clause 7. A drug delivery device of clause 1, wherein the at least one processor and the sensor are positioned on a printed circuit board (PCB).

Clause 8. A drug delivery device of clause 1, further including a housing, and a drug injection system. The at least one processor and the drug injection system are positioned within the housing.

Clause 9. A drug delivery device of clause 8, wherein the drug injection system comprises a cannula configured to be inserted into at least one of a vascular, a dermis, an intradermal tissue, a subcutaneous tissue and a muscle tissue of a user.

Clause 10. A drug delivery device of clause 1, wherein the sensor is a contact sensor, wherein, when receiving the data from the sensor, the at least one processor is programmed or configured to receive data associated with a position of the contact sensor from the contact sensor. When determining the characteristic associated with the sensor, the at least one processor is programmed or configured to determine a condition of the contact sensor with regard to the contact sensor being adjacent a body of a user based on the data associated with a position of the contact sensor. When determining whether the characteristic associated with the sensor satisfies the threshold, the at least one processor is programmed or configured to determine whether the condition of the contact sensor satisfies a threshold value associated with the contact sensor being adjacent the body of the user, and when performing the action, the at least one processor is programmed or configured to perform the action based on determining that the condition of the contact sensor satisfies the threshold value associated with the contact sensor being adjacent the body of the user.

Clause 11. A drug delivery device of clause 1, wherein the threshold includes a threshold value based on a working mode of the drug delivery device.

Clause 12. A drug delivery device including a housing, a drug injection system, a sensor, and at least one processor in communication with the sensor. The at least one processor, the sensor, and the drug injection system are positioned within the housing, and the at least one processor is programmed or configured to receive data associated with the sensor from the sensor, determine a characteristic associated with the sensor based on the data received from the sensor, determine whether the characteristic associated with the sensor satisfies a threshold, and perform an action based on determining that the data received from the sensor satisfies the threshold.

Clause 13. A drug delivery device of clause 12, wherein the sensor is an acceleration sensor, wherein, when receiving the data from the sensor, the at least one processor is programmed or configured to receive data associated with acceleration of the acceleration sensor from the acceleration sensor. When determining the characteristic associated with the sensor, the at least one processor is programmed or configured to determine a velocity of the acceleration sensor based on the data associated with acceleration of the acceleration sensor. When determining whether the characteristic associated with the sensor satisfies the threshold, the at least one processor is programmed or configured to determine whether the velocity of the acceleration sensor satisfies a threshold value of velocity, and when performing the action, the at least one processor is programmed or configured to perform the action based on determining that the velocity of the acceleration sensor satisfies the threshold value of velocity.

Clause 14. A drug delivery device of clause 12, wherein the sensor is an acceleration sensor, wherein, when receiving the data from the sensor, the at least one processor is programmed or configured to receive data associated with acceleration of the acceleration sensor from the acceleration sensor. When determining the characteristic associated with the sensor, the at least one processor is programmed or configured to determine an amount of inertial force of the acceleration sensor based on the data associated with acceleration of the acceleration sensor. When determining whether the characteristic associated with the sensor satisfies the threshold, the at least one processor is programmed or configured to determine whether the amount inertial force of the acceleration sensor satisfies a threshold value of force, and when performing the action, the at least one processor is programmed or configured to perform the action based on determining that the inertial force of the acceleration sensor satisfies the threshold value of force.

Clause 15. A drug delivery device of clause 12, wherein the sensor is an acceleration sensor, wherein, when receiving the data from the sensor, the at least one processor is programmed or configured to receive data associated with acceleration of the acceleration sensor from the acceleration sensor. When determining the characteristic associated with the sensor, the at least one processor is programmed or configured to determine a first vector value of acceleration for the acceleration sensor in a first direction based on the data associated with acceleration of the acceleration sensor, determine a second vector value of acceleration for the acceleration sensor in a second direction based on the data associated with acceleration of the acceleration sensor, and determine a resultant acceleration vector of acceleration for the acceleration sensor based on the first vector value of acceleration of the acceleration sensor in the first direction and the second vector value of acceleration of the acceleration sensor in the second direction. When determining whether the characteristic associated with the sensor satisfies the threshold, the at least one processor is programmed or configured to determine whether the resultant acceleration vector of acceleration for the acceleration sensor satisfies a threshold value of acceleration, and when performing the action, the at least one processor is programmed or configured to perform the action based on determining that the resultant acceleration vector of acceleration for the acceleration sensor satisfies the threshold value of acceleration.

Clause 16. A drug delivery device of clause 12, wherein the sensor is a contact sensor, wherein, when receiving the data from the sensor, the at least one processor is programmed or configured to receive data associated with a position of the contact sensor from the contact sensor. When determining the characteristic associated with the sensor, the at least one processor is programmed or configured to determine a condition of the contact sensor with regard to the contact sensor being adjacent a body of a user based on the data associated with a position of the contact sensor. When determining whether the characteristic associated with the sensor satisfies the threshold, the at least one processor is programmed or configured to determine whether the condition of the contact sensor satisfies a threshold value associated with the contact sensor being adjacent the body of the user, and when performing the action, the at least one processor is programmed or configured to perform the action based on determining that the condition of the contact sensor satisfies the threshold value associated with the contact sensor being adjacent the body of the user.

Clause 17. A drug delivery device of clause 16, wherein the contact sensor comprises at least one of a proximity sensor, an optical sensor, a conduction sensor, or a measurement circuit.

Clause 18. A drug delivery device of clause 17, wherein the proximity sensor is at least one of a capacitive sensor and an ultrasonic sensor.

Clause 19. A drug delivery device including at least one processor programmed or configured to receive data associated with a sensor from the sensor, determine a characteristic associated with the sensor based on the data received from the sensor, determine whether the characteristic associated with the sensor satisfies a threshold, and perform an action based on determining that the data received from the sensor satisfies the threshold. When performing the action, the at least one processor is programmed or configured to perform at least one of provide an alarm, provide a notification, cease delivery of a drug from the drug delivery device, store data associated with an event, communicate data associated with the event to a remote device, or any combination thereof.

Clause 20. A drug delivery device of clause 18, further including a housing, an drug injection system, and an adhesive layer, wherein the at least one processor and the drug injection system are positioned within the housing, and wherein the adhesive layer is configured to be positioned on a body of a user.

Clause 21. A drug delivery device of clause 20, wherein the drug injection system includes a cannula configured to be inserted into at least one of a vascular, a dermis, an intradermal tissue, a subcutaneous tissue and a muscle tissue of a patient.

### BRIEF DESCRIPTION OF THE DRAWINGS

Additional advantages and details are explained in greater detail below with reference to the exemplary embodiments that are illustrated in the accompanying schematic figures, in which:

FIG. 1 is a diagram of a non-limiting embodiment of a drug delivery device;

FIG. 2 is a diagram of a non-limiting embodiment or aspect of components of one or more devices and/or one or more systems of FIG. 1;

FIG. 3 is a flowchart of a non-limiting embodiment of a process for sensing movement of a drug delivery device;

FIG. 4 is a diagram of a non-limiting embodiment of a drug delivery device;

FIG. 5 is a diagram of a non-limiting embodiment of a drug delivery device;

FIG. 6 is a diagram of a non-limiting embodiment of a drug delivery device;

FIG. 7 is a diagram of a non-limiting embodiment of a drug delivery device;

FIG. 8 is a diagram of a non-limiting embodiment of a drug delivery device;

FIG. 9 is a perspective view of a non-limiting embodiment of a drug delivery device;

FIG. 10 is a cross-sectional view of the drug delivery device shown in FIG. 9;

FIG. 11 is a perspective view of the drug delivery device of FIG. 9, with a top cover removed; and

FIG. 12 is a schematic of the drug delivery device of FIG. 9.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to embodiments or aspects as they are oriented in the drawing figures. However, it is to be understood that embodiments or aspects may assume various alternative variations and step sequences, except where expressly specified to the contrary. It is also to be understood that the specific devices and processes illustrated in the attached drawings, and described in the following specification, are simply non-limiting exemplary embodiments or aspects. Hence, specific dimensions and other physical characteristics related to the embodiments or aspects of the embodiments or aspects disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” As used in the specification and the claims, the singular form of “a,” “an,” and “the” include plural referents, such as unless the context clearly dictates otherwise. Additionally, Furthermore, as used herein, the terms “set” and “group” are intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. Further, the phrase “based on” is intended to mean “based at least partially on” unless explicitly stated otherwise. Further, the phrase “based on” may mean “in response to” and be indicative of a condition for automatically triggering a specified operation of an electronic device (e.g., a controller, a processor, a computing device, etc.) as appropriately referred to herein.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or send (e.g., transmit) information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and transmits the processed information to the second unit. In some non-limiting embodiments or aspects, a message may refer to a network packet (e.g., a data packet and/or the like) that includes data.

Some non-limiting embodiments or aspects may be described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

Provided are improved devices, systems, methods and computer program products for sensing movement of a drug delivery device. Embodiments of the present disclosure may include a drug delivery device that includes at least one sensor and at least one processor in communication with the sensor, wherein the at least one processor is programmed or configured to receive data associated with the sensor from the sensor, determine a characteristic associated with the sensor based on the data received from the sensor, determine whether the characteristic associated with the sensor satisfies a threshold, and perform an action based on determining that the data received from the sensor satisfies the threshold. In some non-limiting embodiments, when performing the action, the at least one processor is programmed or configured to perform an action such as provide a notification to a user, request an action be performed, such as reposition the device, stop delivery and/or provide an alarm. The processor may also be configured to provide additional functionality, such as causing a stop to delivery of a drug, providing a status signal, communicate wirelessly with a handheld device or smartphone, and/or store data associated with an event.

In certain configurations, the drug delivery device may include, for example, an accelerometer and a second sensor, such as a pressure sensor which may indicate leakage has occurred as a result of movement dislodging a catheter or cannula.

In some non-limiting embodiments, the threshold comprises a threshold value based on a working mode of the drug delivery device. In some non-limiting embodiments, the at least one processor is further programmed or configured to determine an orientation of the sensor, and, when determining the characteristic associated with the sensor, the at least one processor is programmed or configured to determine the characteristic associated with the sensor based on the orientation of the sensor. In some non-limiting embodiments, the at least one processor and the sensor are positioned on a printed circuit board (PCB). In some non-limiting embodiments, the drug delivery device further comprises a housing and a drug injection system, and the at least one processor and the drug injection system are positioned within the housing. In some non-limiting embodiments, the drug injection system comprises a cannula configured to be inserted into dermis, subcutaneous tissue or muscle tissue of a user, and/or the patient's vasculature (e.g., a patient).

In this way, the present disclosure allows for a wearable drug delivery device that determines an event, such as shock, acceleration, movement, and/or detachment) that may lead to a mis-positioning of the device after application to a patient and before the end of drug delivery to the patient.

In other embodiments of the present disclosure allow for a wearable drug delivery device that may determine when the drug delivery device is properly positioned on a body of a user to ensure correct delivery of a drug to the user.

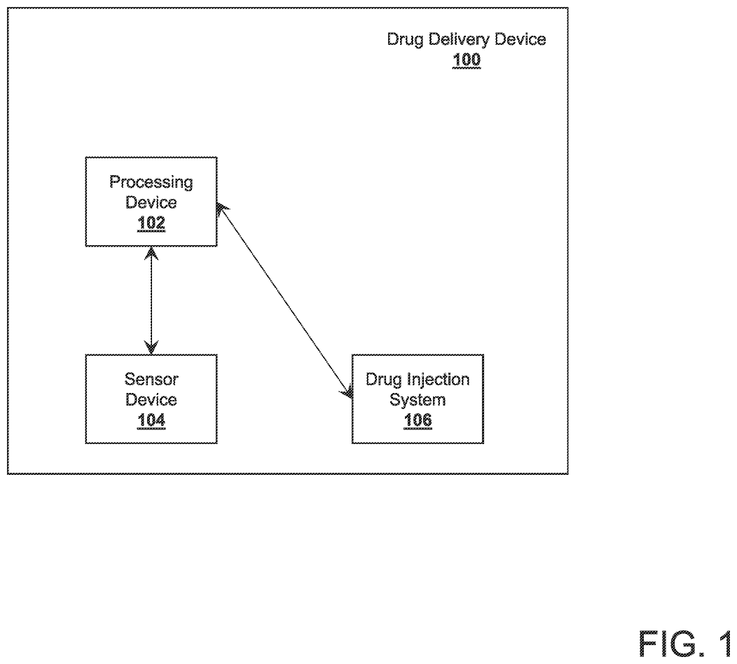

Referring now to FIG. 1, FIG. 1 is a diagram of drug delivery device 100. As shown in FIG. 1, drug delivery device 100 may include processing device 102, sensor device 104, and drug injection system 106. Processing device 102, sensor device 104, and drug injection system 106 may interconnect (e.g., establish a connection to communicate, and/or the like) via wired connections, wireless connections, or a combination of wired and wireless connections.

In some non-limiting embodiments, drug delivery device 100 may be a wearable automatic injector, such as an insulin or bone marrow stimulant delivery device. For example, drug delivery device 100 may be applied to the skin of a user and triggered to inject a drug into the user. Drug delivery device 100 may be pre-filled with the drug, or it may be filled (e.g., refilled following a prior injection) with the drug by the user or another individual (e.g., a medical professional) prior to use.

Drug delivery device 100 may be configured to deliver a dose of a drug into the body of a user by injection at a controlled rate of delivery. The controlled rate of delivery may include time durations for the delivery from about 5 minutes to about 60 minutes, but are not limited to this exemplary range. Exemplary volumes of the drug delivered by drug delivery device 100 may range from about 0.1 milliliters to about 10 milliliters, but are not limited to this range. The volume of the drug delivered to the user may be adjusted (e.g., atomically adjusted or manually adjusted).

In some non-limiting embodiments, processing device 102 may include a device that is capable of being in communication with sensor device 104 and/or drug injection system 106. In some non-limiting embodiments, processing device 102 may include a device controller (e.g., a microcontroller), a processor, an integrated circuit, and/or the like. In some non-limiting embodiments, processing device 102 may provide commands (e.g., instructions, signals, etc.) to drug injection system 106 to cause drug injection system 106 to deliver a drug to a body of a user (e.g., a patient) upon which drug delivery device 100 is applied.

In some non-limiting embodiments, sensor device 104 may include a device that is capable of being in communication with processing device 102. In some non-limiting embodiments, sensor device 104 may include at least one sensor, such as an acceleration sensor and/or a contact sensor. In some embodiments, a second or additional sensor may also be utilized, such as a pressure sensor, to help determine if leakage or another event has occurred which may lead, or has led, to the mis-positioning of the drug delivery device. In some non-limiting embodiments, the acceleration sensor may include an accelerometer, such as a three-axis accelerometer. In some non-limiting embodiments, the three-axis accelerometer may be capable of detecting acceleration in each direction of a three dimensional (e.g., xyz) coordinate system. Additionally, the three-axis accelerometer may be capable of detecting an angular rate of acceleration in each direction of the three dimensional coordinate system. In some non-limiting embodiments, the contact sensor may include a sensor capable of detecting contact with a body of a user. For example, the contact sensor may include a proximity sensor, an optical sensor, a conduction sensor, and/or a circuit for measuring a property (e.g., a measurement circuit). In some non-limiting embodiments, the measurement circuit may be an anti-tampering circuit. In some non-limiting embodiments, sensor device 104 may provide data associated with sensor device 104 based on sensor device 104 measuring a characteristic of sensor device 104 and/or an environment of sensor device 104.

In some non-limiting embodiments, drug injection system 106 may include a device that is capable of being in communication with processing device 102. In some non-limiting embodiments, drug injection system 106 may include a device (e.g., a mechanism, a structure, etc.) that is capable of delivering a drug to a body of a user upon which drug delivery device 100 is applied. In some non-limiting embodiments, drug injection system 106 may automatically deliver the drug to the body of the user according to one or more predetermined time intervals. Additionally or alternatively, drug injection system 106 may automatically deliver the drug to the body of the user at a predetermined time period, such as at a predetermined time period following application of drug delivery device 100 to the body of the user. In some non-limiting embodiments, drug injection system 106 may include a cannula, a catheter, and/or a needle that is configured to be positioned within the body of the user. For example, drug injection system 106 may include a cannula, a catheter, and/or a needle that is configured to be inserted into the dermis, subcutaneous tissue or muscle tissue of the user.

In some non-limiting embodiments, processing device 102, sensor device 104, and/or drug injection system 106 may be positioned on (e.g., installed on, placed on, integrated with, etc.) a printed circuit board (PCB) of drug delivery device 100. For example, processing device 102, sensor device 104, and/or drug injection system 106 may be positioned on the same PCB. In some non-limiting embodiments, the PCB that has processing device 102, sensor device 104, and/or drug injection system 106 positioned thereon, may be positioned within a housing of drug delivery device 100. In this way, drug delivery device 100 may have a compact form factor that allows for a user to wear drug delivery device 100 with minimal discomfort.

In some non-limiting embodiments, drug delivery device 100 may include a mechanism for holding drug delivery device 100 in contact with a body of a user. In this way, drug delivery device 100 may be a body worn device. In one example, drug delivery device 100 may include an adhesive layer that has an adhesive appropriate for holding drug delivery device 100 on the body of the user for an extend time period. Additionally or alternatively, drug delivery device 100 may include a band (e.g., a strap, a belt, a wrap, etc.) configured to hold drug delivery device 100 housing in contact with the body of the user. In some non-limiting embodiments, the adhesive layer may be positioned on a surface of drug delivery device 100 that is positioned near the body of the user when the drug delivery device 100 is applied to the body of the user. Further, a component of drug injection system 106 may be configured to extend through the adhesive layer to be positioned within the body of the user. In some non-limiting embodiments, the component of drug injection system 106 may be configured to extend through an aperture of the adhesive layer. In some non-limiting embodiments, the component of drug injection system 106 may be configured to penetrate through the adhesive layer.

In some non-limiting embodiments, drug delivery device 100 may include a device for providing an indication associated with an operation of drug delivery device 100. For example, drug delivery device 100 may include one or more devices that provide a visual prompt to a user, such as one or more light emitting diodes (LEDS) and/or one or more display screens. Additionally or alternatively, drug delivery device 100 may include one or more devices that provide an audible prompt to a user, such as one or more electroacoustic transducer (e.g., one or more speakers).

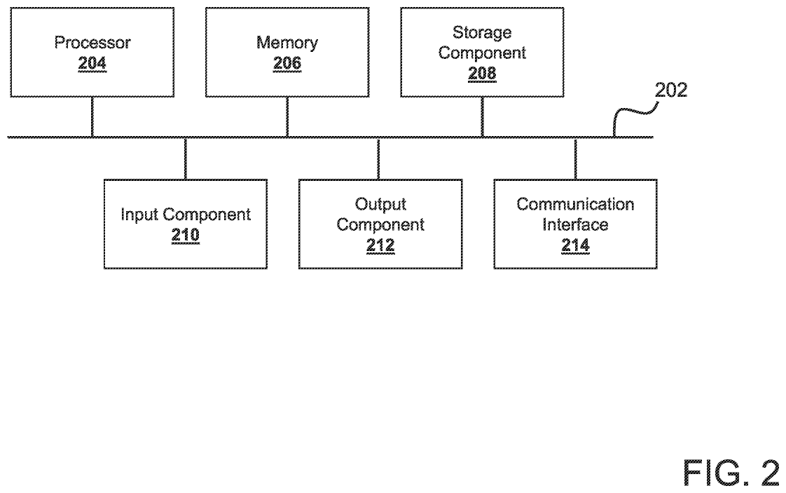

Referring now to FIG. 2, FIG. 2 is a diagram of example components of device 200. Device 200 may correspond to processing device 102, sensor device 104, and/or one or more components of drug injection system 106. In some non-limiting embodiments, processing device 102, sensor device 104, and/or drug injection system 106 may include at least one device 200 and/or at least one component of device 200. As shown in FIG. 2, device 200 may include bus 202, processor 204, memory 206, storage component 208, input component 210, output component 212, and communication interface 214.

Bus 202 may include a component that permits communication among the components of device 200. In some non-limiting embodiments or aspects, processor 204 may be implemented in hardware, software, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 may include random access memory (RAM), read-only memory (ROM), and/or another type of dynamic or static storage device (e.g., flash memory, magnetic memory, optical memory, etc.) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touchscreen display, a keyboard, a keypad, a mouse, a button, a switch, a microphone, a camera, etc.). Additionally or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a Bluetooth® interface, a Zigbee® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally or alternatively, hardwired circuitry may be used in place of or in combination with software instructions to perform one or more processes described herein. Thus, embodiments or aspects described herein are not limited to any specific combination of hardware circuitry and software.

Memory 206 and/or storage component 208 may include data storage or one or more data structures (e.g., a database and/or the like). Device 200 may be capable of receiving information from, storing information in, communicating information to, or searching information stored in the data storage or one or more data structures in memory 206 and/or storage component 208. For example, the information may include input data, input data, output data, transaction data, account data, or any combination thereof.

The number and arrangement of components shown in FIG. 2 are provided as an example. In some non-limiting embodiments or aspects, device 200 may include additional components, fewer components, different components, or differently arranged components than those shown in FIG. 2. Additionally or alternatively, a set of components (e.g., one or more components) of device 200 may perform one or more functions described as being performed by another set of components of device 200.

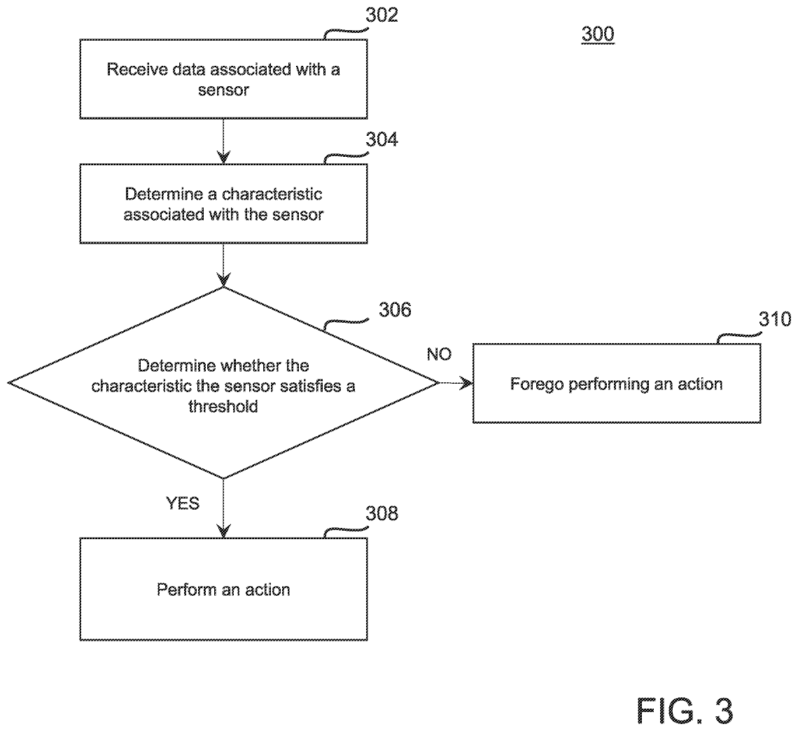

Referring now to FIG. 3, FIG. 3 is a flowchart of a non-limiting aspect or embodiment of process 300 for sensing movement of a drug delivery device. In some non-limiting embodiments or aspects, one or more of the functions described with respect to process 300 may be performed (e.g., completely, partially, etc.) by processing device 102. In some non-limiting embodiments or aspects, one or more of the steps of process 300 may be performed (e.g., completely, partially, and/or the like) by another device or a group of devices separate from and/or including processing device 102, such as sensor device 104 and/or drug injection system 106.

As shown in FIG. 3, at step 302, process 300 may include receiving data associated with a sensor. For example, processing device 102 may receive the data associated with sensor device 104 from sensor device 104. In some non-limiting embodiments, the data associated with sensor device 104 may include any information (e.g., a characteristic, a parameter, etc.) that is determined (e.g., measured, detected, sensed, obtained, etc.) by sensor device 104 in the course of operation of sensor device 104.

As shown in FIG. 3, at step 304, process 300 may include determining a characteristic associated with the sensor. For example, processing device 102 may determine a characteristic associated with sensor device 104 based on the data received from sensor device 104. In some non-limiting embodiments, processing device 102 may determine an orientation of sensor device 104. In some non-limiting embodiments, processing device 102 may calibrate sensor device 104 based on the orientation of sensor device 104. In some non-limiting embodiments, processing device 102 may determine the characteristic associated with the sensor based on the orientation of sensor device 104 and/or a calibration of sensor device 104.

As shown in FIG. 3, at step 306, process 300 may include determining whether the characteristic associated with the sensor satisfies a threshold. For example, processing device 102 may determine whether the characteristic associated with sensor device 104 satisfies a threshold. In some non-limiting embodiments, the threshold may include a threshold value that is based on an amount of force. In one example, the threshold may include a threshold value that is based on a peel force required to cause movement of drug delivery device 100 in specific direction. In another example, the threshold may include a threshold value that is based on an amount of force that causes skin deformation leading to movement of a cannula, a catheter, and/or a needle of drug injection system 106. In such an example, the threshold value may be based on an amount of force that causes skin deformation leading to movement of a cannula, a catheter, and/or a needle of drug injection system 106 without peeling of an adhesive that holds drug delivery device 100 to the body of a user. In some non-limiting embodiments, the threshold may include a threshold value that is based on a working mode of drug delivery device 100. Drug delivery device 100 may have a plurality of working modes including a mode associated with when a user is asleep (e.g., deep sleep), a working mode associated with preparation of applying drug delivery device 100 to a body of a user, a working mode associated with deployment of a component (e.g., a cannula, a catheter, and/or a needle) of drug injection system 106, and/or a working mode associated with an injection of a drug (e.g., a time at which the drug is being delivered) to the body of the user. In some non-limiting embodiments, the threshold value may be based on the working mode, such that a threshold value for a first working mode may be different than a threshold value for a second working mode. In some non-limiting embodiments, the threshold value may be different for each working mode of the plurality of working modes of drug delivery device 100.

As shown in FIG. 3, at step 308 (“YES”), process 300 may include performing an action based on determining that the characteristic associated with the sensor satisfies the threshold. For example, processing device 102 may perform an action based on determining that the characteristic associated with sensor device 104 satisfies the threshold. In some non-limiting embodiments, processing device 102 may compare the characteristic associated with sensor device 104 to the threshold (e.g., a threshold value), if processing device 102 determines that the characteristic associated with sensor device 104 satisfies the threshold, processing device 102 may perform the action. In some non-limiting embodiments, processing device 102 may perform the action by providing a notification to a user, requesting that an action be performed, repositioning the device, stopping delivery, and/or providing an alarm.

For example, processing device 102 may cause (e.g., trigger) an alarm to be emitted by drug delivery device 100. In some non-limiting embodiments, processing device 102 may perform the action by providing a notification. For example, processing device 102 may cause a notification (e.g., a notification based on a visual prompt, such as a light emitting diode being illuminated, a notification based on an audible prompt, such as a speaker emitting an audible sound, etc.) to be provided by drug delivery device 100. In some non-limiting embodiments, processing device 102 may perform the action by storing (e.g., logging) data associated with an event (e.g., an event indicating that the characteristic associated with sensor device 104 satisfies a threshold). For example, processing device 102 may store data associated with the event in a memory device of drug delivery device 100.

As shown in FIG. 3, at step 310 (“NO”), process 300 may include foregoing performing an action based on determining that the characteristic associated with the sensor does not satisfy the threshold. For example, processing device 102 may forego performing an action based on determining that the characteristic associated with sensor device 104 does not satisfy the threshold. In some non-limiting embodiments, processing device 102 may compare the characteristic associated with sensor device 104 to the threshold, if processing device 102 determines that the characteristic associated with sensor device 104 does not satisfy the threshold, processing device 102 may forego performing the action.

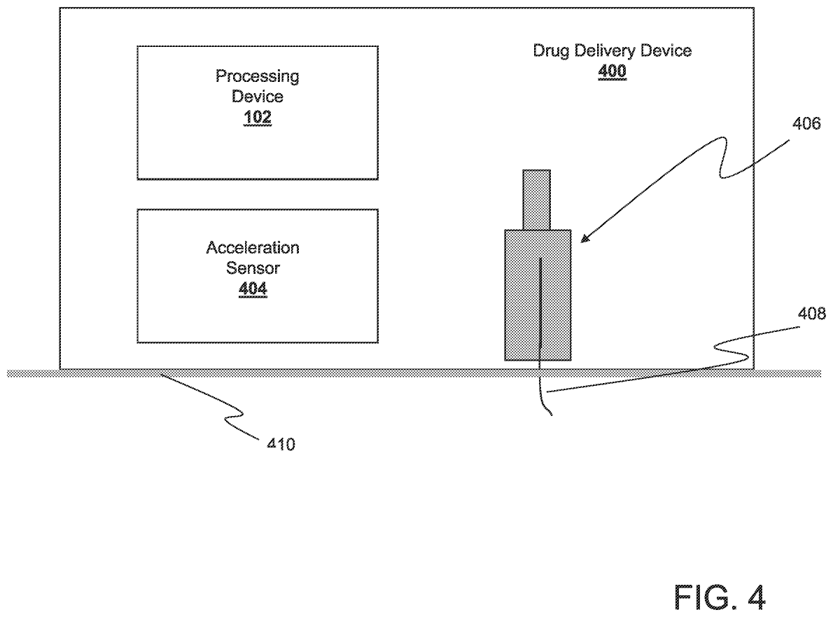

With regard to FIGS. 4-8, processing device 102 may be programmed or configured to perform a process as appropriate based on a specific type of sensor of a drug delivery device, of which processing device 102 is a component. In some non-limiting embodiments, processing device 102 may be programed or configured to perform a process that is the same or similar to process 300 for sensing movement of a drug delivery device.

Referring now to FIG. 4, FIG. 4 is a diagram of drug delivery device 400. In some non-limiting embodiments, drug delivery device 400 may be the same as or similar to drug delivery device 100. As shown in FIG. 4, drug delivery device 400 may include processing device 102, acceleration sensor 404, drug injection system 406, and adhesive layer 410. In some non-limiting embodiments, acceleration sensor 404 may be the same as or similar to sensor device 104. In some non-limiting embodiments, acceleration sensor 404 may include an accelerometer. For example, acceleration sensor 404 may include a three-axis accelerometer (e.g., a three-axis gyroscope). In some non-limiting embodiments, drug injection system 406 may include cannula 408. In some non-limiting embodiments, cannula 408 may include a needle. In some non-limiting embodiments, cannula 408 may include a catheter, which may be a soft, flexible catheter, a rigid catheter, or a combination thereof. In some non-limiting embodiments, adhesive layer 410 may include a structural layer (e.g., a base, a baseplate, etc.) and an adhesive applied on the structural layer. In some non-limiting embodiments, the structural layer may include a flexible polymeric foam, a woven material, a hard plastic material, and/or the like. In some non-limiting embodiments, adhesive layer 410 may come into contact with the body of a user. For example, adhesive layer 410 may be applied to the skin of a user. In some non-limiting embodiments, cannula 408 and/or a needle of cannula 408 may be inserted into the skin of a user.

With regard to drug delivery device 400, processing device 102 may be programed or configured to perform a process that is the same or similar to process 300 for sensing movement of a drug delivery device based on acceleration sensor 404. In some non-limiting embodiments, processing device 102 may receive data associated with acceleration of acceleration sensor 404 from acceleration sensor 404.

In some non-limiting embodiments, processing device 102 may determine a characteristic associated with acceleration sensor 404 based on the data associated with acceleration of acceleration sensor 404. In some non-limiting embodiments, processing device 102 may determine a velocity of the acceleration sensor based on the data associated with acceleration of acceleration sensor 404. For example, processing device 102 may calculate a value of velocity based on a measured value of acceleration included in the data associated with acceleration of acceleration sensor 404. In some non-limiting embodiments, processing device 102 may determine an amount of inertial force of acceleration sensor 404 based on the data associated with acceleration of acceleration sensor 404. In some non-limiting embodiments, when determining the amount of inertial force of acceleration sensor 404, processing device 102 is programmed or configured to determine a first amount of inertial force in a first direction (e.g., one of an x direction, a y direction, or a z direction in an xyz coordinate system) based on first data associated with acceleration of the acceleration sensor in a first direction and determine a second amount of inertial force in a second direction based on second data associated with acceleration of the acceleration sensor in a second direction. In some non-limiting embodiments, processing device 102 may determine a first vector value of acceleration of the acceleration sensor in a first direction based on the data associated with acceleration of the acceleration sensor, determine a second vector value of acceleration of the acceleration sensor in a second direction based on the data associated with acceleration of the acceleration sensor, and determine a resultant acceleration vector for the acceleration sensor based on the first vector value of acceleration of the acceleration sensor in the first direction and the second vector value of acceleration of the acceleration sensor in the second direction.

In some non-limiting embodiments, processing device 102 may determine whether the characteristic associated with acceleration sensor 404 satisfies a threshold value based on the characteristic associated with acceleration sensor 404. In some non-limiting embodiments, processing device 102 may determine whether the velocity of the acceleration sensor satisfies a threshold value of velocity. In some non-limiting embodiments, processing device 102 may determine whether an amount of inertial force of acceleration sensor 404 satisfies a threshold value of force. In some non-limiting embodiments, when determining whether the inertial force of acceleration sensor 404 satisfies a threshold value of force, processing device 102 may determine whether a first amount of inertial force in a first direction satisfies a first threshold value of force and whether a second amount of inertial force in a second direction satisfies a second threshold value of force. In some non-limiting embodiments, processing device 102 may determine whether a resultant acceleration vector for acceleration sensor 404 satisfies a threshold value of acceleration.

In some non-limiting embodiments, processing device 102 may perform an action based on determining that the characteristic associated with acceleration sensor 404 satisfies the threshold value or forego performing an action based on determining that the characteristic associated with acceleration sensor 404 does not satisfy the threshold value. In some non-limiting embodiments, processing device 102 may perform the action based on determining that the velocity of acceleration sensor 404 satisfies the threshold value of velocity or processing device 102 may forego performing the action based on determining that the velocity of acceleration sensor 404 does not satisfy the threshold value of velocity. In some non-limiting embodiments, processing device 102 may perform the action based on determining that the inertial force of acceleration sensor 404 satisfies the threshold value of force or forego performing the action based on determining that the inertial force of acceleration sensor 404 does not satisfy the threshold value of force. In some non-limiting embodiments, when performing the action, processing device 102 may perform the action based on determining that the first amount of inertial force in the first direction satisfies the first threshold value of force and/or that the second amount of inertial force in the second direction satisfies the second threshold value of force. In some non-limiting embodiments, when foregoing performing the action, processing device 102 may forego performing the action based on determining that the first amount of inertial force in the first direction does not satisfy the first threshold value of force and/or that the second amount of inertial force in the second direction does not satisfy the second threshold value of force. In some non-limiting embodiments, processing device 102 may perform the action based on determining that the resultant acceleration vector of acceleration for acceleration sensor 404 satisfies the threshold value of acceleration or forego performing the action based on determining that the resultant acceleration vector of acceleration for acceleration sensor 404 does not satisfy the threshold value of acceleration.

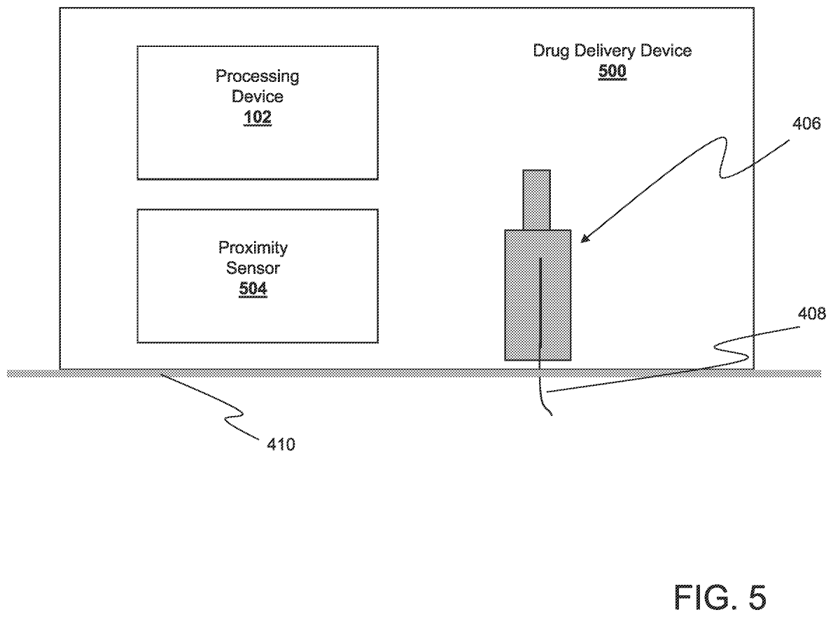

With regard to each of FIGS. 5-8, as shown in FIGS. 5-8, sensor device 104 has been replaced with a type of contact sensor, such as proximity sensor 504, optical sensor 604, conduction sensor 704, and measurement circuit 804 with electrical trace 814, for detecting whether a drug delivery device is in contact with a body of a user. With regard to drug delivery device 500, drug delivery device 600, drug delivery device 700, and drug delivery device 800, processing device 102 may be programed or configured to perform a process that is the same or similar to process 300 for sensing movement of the drug delivery device based on a contact sensor (e.g., proximity sensor 504, optical sensor 604, conduction sensor 704, and/or measurement circuit 804 with electrical trace 814).

In some non-limiting embodiments, processing device 102 may receive data associated with a position of a contact sensor (e.g., proximity sensor 504, optical sensor 604, conduction sensor 704, or measurement circuit 804 with electrical trace 814) from the contact sensor. In some non-limiting embodiments, the data associated with the position of the contact sensor may include an indication of whether the contact sensor is adjacent (e.g., in contact with, in proximity to, within a predetermined distance of, etc.) a body of the user. In some non-limiting embodiments, processing device 102 may determine a characteristic associated with the contact sensor. For example, processing device 102 may determine a condition of the contact sensor with regard to the contact sensor being adjacent to a body of a user (e.g., a condition associated with the contact sensor being or not being in contact with a body of the user, a condition associated with the contact sensor being or not being in proximity to a body of the user, a condition associated with the contact sensor being within or not being within a predetermined distance of a body of the user, etc.) based on the data associated with the position of the contact sensor. In some non-limiting embodiments, processing device 102 may determine whether the contact sensor is adjacent to the body of the user based on the data associated with the position of the contact sensor.

In some non-limiting embodiments, processing device 102 may determine whether the characteristic associated with the contact sensor satisfies a threshold. For example, processing device 102 may determine whether the condition of the contact sensor satisfies a threshold value associated with the contact sensor being adjacent the body of the user. In some non-limiting embodiments, the threshold value includes an indication that the contact sensor is adjacent the body of the user. In some non-limiting embodiments, processing device 102 may perform an action based on determining that the condition of the contact sensor satisfies the threshold or forego performing an action based on determining that the condition of the contact sensor does not satisfy the threshold.

Referring now to FIG. 5, FIG. 5 is a diagram of drug delivery device 500. In some non-limiting embodiments, drug delivery device 500 may be the same as or similar to drug delivery device 100 and/or drug delivery device 400. As shown in FIG. 5, drug delivery device 500 may include processing device 102, proximity sensor 504, drug injection system 406, and adhesive layer 410. In some non-limiting embodiments, proximity sensor 504 may be the same as or similar to sensor device 104. In some non-limiting embodiments, proximity sensor 504 may include a device configured to detect whether proximity sensor is adjacent (e.g., in proximity to) the body of the user.

In some non-limiting embodiments, proximity sensor 504 may include a device configured to detect when proximity sensor 504 is within a predetermined threshold of distance to a surface, such as a body (e.g., an appendage of a body) of a user and/or the skin of the user. In some non-limiting embodiments, proximity sensor 504 may include a capacitive proximity sensor, an inductive proximity sensor, an ultrasonic proximity sensor, an optical proximity sensor, and/or the like.

In some non-limiting embodiments, proximity sensor 504 may be adjacent the body of a user and proximity sensor 504 may provide a signal to processing device 102 that indicates that proximity sensor 504 is adjacent the body of the user. In some non-limiting embodiments, proximity sensor 504 may not be adjacent the body of a user and proximity sensor 504 may provide a signal to processing device 102 that indicates that proximity sensor 504 is not adjacent the body of the user or proximity sensor 504 may not provide a signal to processing device 102. In some non-limiting embodiments, processing device 102 may determine that proximity sensor 504 is adjacent the body of the user based on a signal received from proximity sensor 504 to processing device 102. In some non-limiting embodiments, processing device 102 may determine that proximity sensor 504 is not adjacent the body of the user based on a signal received from proximity sensor 504 to processing device 102 or based on failing to receive a signal from proximity sensor 504. In some non-limiting embodiments, processing device 102 may perform an action based on determining that proximity sensor 504 is not adjacent the body of the user. In some non-limiting embodiments, processing device 102 may prevent an operation (e.g., activation) of drug injection system 406 based on determining that proximity sensor 504 is not adjacent the body of the user. In some non-limiting embodiments, processing device 102 may allow for an operation (e.g., an operation associated with delivering a drug to the user, such as an injection) of drug injection system 406 based on determining that proximity sensor 504 is adjacent the body of the user.

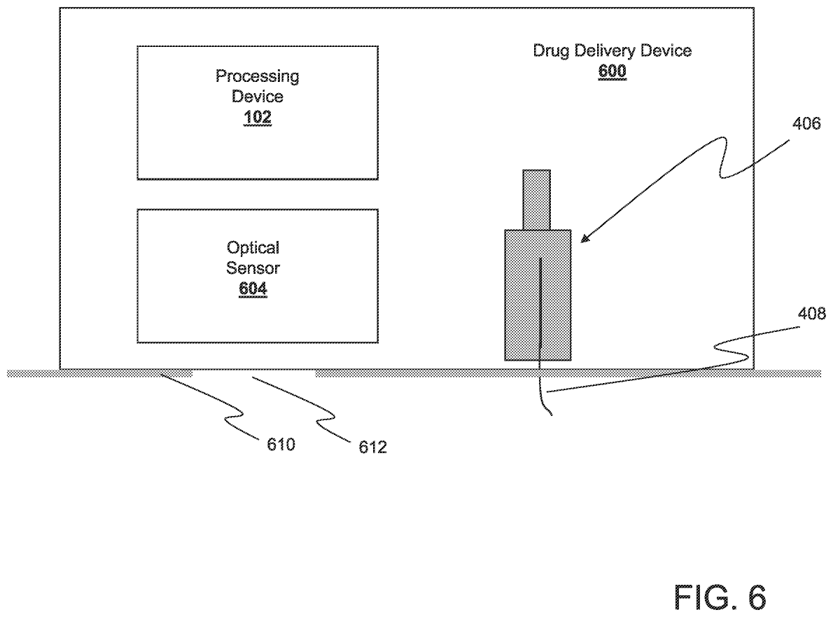

Referring now to FIG. 6, FIG. 6 is a diagram of drug delivery device 600. In some non-limiting embodiments, drug delivery device 600 may be the same as or similar to drug delivery device 100, drug delivery device 400, and/or drug delivery device 500. As shown in FIG. 6, drug delivery device 600 may include processing device 102, optical sensor 604, drug injection system 406, and adhesive layer 610. In some non-limiting embodiments, optical sensor 604 may be the same as or similar to sensor device 104. In some non-limiting embodiments, adhesive layer 610 may be the same as or similar to adhesive layer 410.

In some non-limiting embodiments, optical sensor 604 may include a device configured to detect when optical sensor 604 is within a predetermined threshold of distance to a surface, such as a body (e.g., an appendage of a body) of a user and/or the skin of the user based on light transmitted by and received by (e.g., received based on a reflection) optical sensor 604. In some non-limiting embodiments, optical sensor 604 may include an ambient light optical sensor, an infrared (IR) optical sensor, an ultraviolet optical sensor, and/or the like. In some non-limiting embodiments, optical sensor 604 may include an emitter (e.g., a light emitter) and/or a receiver for receiving the light emitted by the emitter. As shown in FIG. 6, adhesive layer 610 may include aperture 612 that allows for light emitted by optical sensor 604 to contact a body of a user and reflect back and be received by optical sensor 604.

In some non-limiting embodiments, optical sensor 604 may be adjacent the body of a user and optical sensor 604 may provide a signal to processing device 102 that indicates that optical sensor 604 is adjacent the body of the user. In some non-limiting embodiments, optical sensor 604 may not be adjacent the body of a user and optical sensor 604 may provide a signal to processing device 102 that indicates that optical sensor 604 is not adjacent the body of the user or optical sensor 604 may not provide a signal to processing device 102. In some non-limiting embodiments, processing device 102 may determine that optical sensor 604 is adjacent the body of the user based on a signal received from optical sensor 604 to processing device 102. In some non-limiting embodiments, processing device 102 may determine that optical sensor 604 is not adjacent the body of the user based on a signal received from optical sensor 604 to processing device 102 or based on failing to receive a signal from optical sensor 604. In some non-limiting embodiments, processing device 102 may perform an action based on determining that optical sensor 604 is not adjacent the body of the user. In some non-limiting embodiments, processing device 102 may prevent an operation (e.g., activation) of drug injection system 406 based on determining that optical sensor 604 is not adjacent the body of the user. In some non-limiting embodiments, processing device 102 may allow for an operation (e.g., an operation associated with delivering a drug to the user, such as an injection) of drug injection system 406 based on determining that optical sensor 604 is adjacent the body of the user.

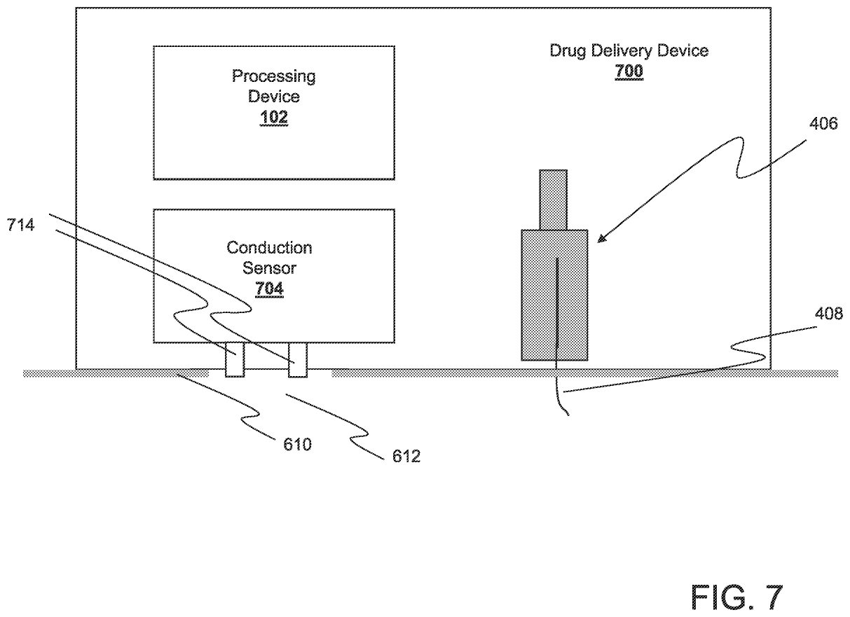

Referring now to FIG. 7, FIG. 7 is a diagram of drug delivery device 700. In some non-limiting embodiments, drug delivery device 700 may be the same as or similar to drug delivery device 100, drug delivery device 400, drug delivery device 500, and/or drug delivery device 600. As shown in FIG. 7, drug delivery device 700 may include processing device 102, conduction sensor 704, drug injection system 406, and adhesive layer 610. In some non-limiting embodiments, conduction sensor 704 may be the same as or similar to sensor device 104.

In some non-limiting embodiments, conduction sensor 704 may include a device configured to detect when conduction sensor 704 is in contact with a surface, such as a body (e.g., skin on a body) of a user. In some non-limiting embodiments, conduction sensor 704 may include a device that measures an electrical property (e.g., current, voltage, resistance, etc.) and determines whether conduction sensor 704 is in contact with the body of the user. In some non-limiting embodiments, conduction sensor 704 may include a piezoelectric sensor, an ohmmeter, a voltmeter, a current sensor, and/or the like. As further shown in FIG. 7, conduction sensor 704 may include electrical contacts 714. As shown in FIG. 7, adhesive layer 610 may include aperture 612 that allows electrical contacts 714 of conduction sensor 704 to contact a body of a user.

In some non-limiting embodiments, electrical contacts 714 of conduction sensor 704 may be in contact with the body of a user and conduction sensor 704 may provide a signal to processing device 102 that indicates that conduction sensor 704 is adjacent the body of the user. In some non-limiting embodiments, electrical contacts 714 of conduction sensor 704 may not be in contact with the body of the user and conduction sensor 704 may provide a signal to processing device 102 that indicates that conduction sensor 704 is not adjacent the body of the user or conduction sensor 704 may not provide a signal to processing device 102. In some non-limiting embodiments, processing device 102 may determine that conduction sensor 704 is adjacent the body of the user based on a signal received from conduction sensor 704 to processing device 102. In some non-limiting embodiments, processing device 102 may determine that conduction sensor 704 is not adjacent the body of the user based on a signal received from conduction sensor 704 to processing device 102 or based on failing to receive a signal from conduction sensor 704. In some non-limiting embodiments, processing device 102 may perform an action based on determining that conduction sensor 704 is not adjacent the body of the user. In some non-limiting embodiments, processing device 102 may prevent an operation (e.g., activation) of drug injection system 406 based on determining that conduction sensor 704 is not adjacent the body of the user. In some non-limiting embodiments, processing device 102 may allow for an operation (e.g., an operation associated with delivering a drug to the user, such as an injection) of drug injection system 406 based on determining that conduction sensor 704 is adjacent the body of the user.

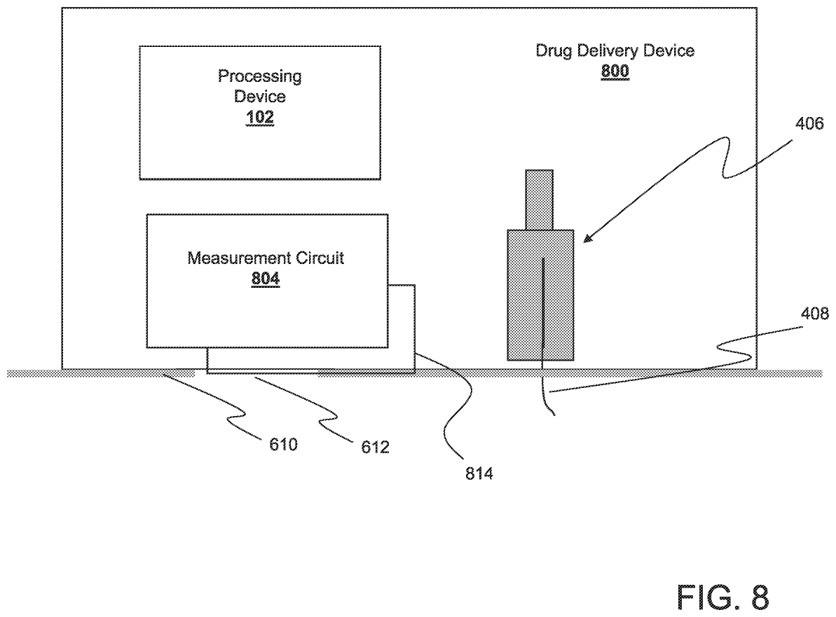

Referring now to FIG. 8, FIG. 8 is a diagram of drug delivery device 800. In some non-limiting embodiments, drug delivery device 800 may be the same as or similar to drug delivery device 100, drug delivery device 400, drug delivery device 500, drug delivery device 600, and/or drug delivery device 700. As shown in FIG. 8, drug delivery device 800 may include processing device 102, measurement circuit 804, drug injection system 406, and adhesive layer 610. In some non-limiting embodiments, measurement circuit 804 may be the same as or similar to sensor device 104. As further shown in FIG. 8, measurement circuit 804 may include conductive trace 814. As further shown in FIG. 8, adhesive layer 610 may include aperture 612 that allows conductive trace 814 of measurement circuit 804 to contact a body of a user.

In some non-limiting embodiments, measurement circuit 804 may include a device configured to detect when conductive trace 814 is in contact with a surface, such as a body (e.g., skin on a body) of a user. In some non-limiting embodiments, measurement circuit 804 may include a device that measures an electrical property (e.g., current, voltage, resistance, etc.) and determines whether conductive trace 814 is in contact with the body of the user. In some non-limiting embodiments, measurement circuit 804 may include a device that measures a physical property (e.g., strain) and determines whether conductive trace 814 is in contact with the body of the user. In some non-limiting embodiments, measurement circuit 804 may include a piezoelectric sensor, an ohmmeter, a voltmeter, a current sensor, a strain gauge, and/or the like. In some non-limiting embodiments, conductive trace 814 may be positioned within adhesive layer 610.

In some non-limiting embodiments, conductive trace 814 of measurement circuit 804 may be in contact with the body of a user and measurement circuit 804 may provide a signal to processing device 102 that indicates that measurement circuit 804 is adjacent the body of the user. In some non-limiting embodiments, conductive trace 814 of measurement circuit 804 may not be in contact with the body of the user and measurement circuit 804 may provide a signal to processing device 102 that indicates that measurement circuit 804 is not adjacent the body of the user or measurement circuit 804 may not provide a signal to processing device 102. In some non-limiting embodiments, processing device 102 may determine that measurement circuit 804 is adjacent the body of the user based on a signal received from measurement circuit 804 to processing device 102. In some non-limiting embodiments, processing device 102 may determine that measurement circuit 804 is not adjacent the body of the user based on a signal received from measurement circuit 804 to processing device 102 or based on failing to receive a signal from measurement circuit 804. In some non-limiting embodiments, processing device 102 may perform an action based on determining that measurement circuit 804 is not adjacent the body of the user. In some non-limiting embodiments, processing device 102 may prevent an operation (e.g., activation) of drug injection system 406 based on determining that measurement circuit 804 is not adjacent the body of the user. In some non-limiting embodiments, processing device 102 may allow for an operation (e.g., an operation associated with delivering a drug to the user, such as an injection) of drug injection system 406 based on determining that measurement circuit 804 is adjacent the body of the user.

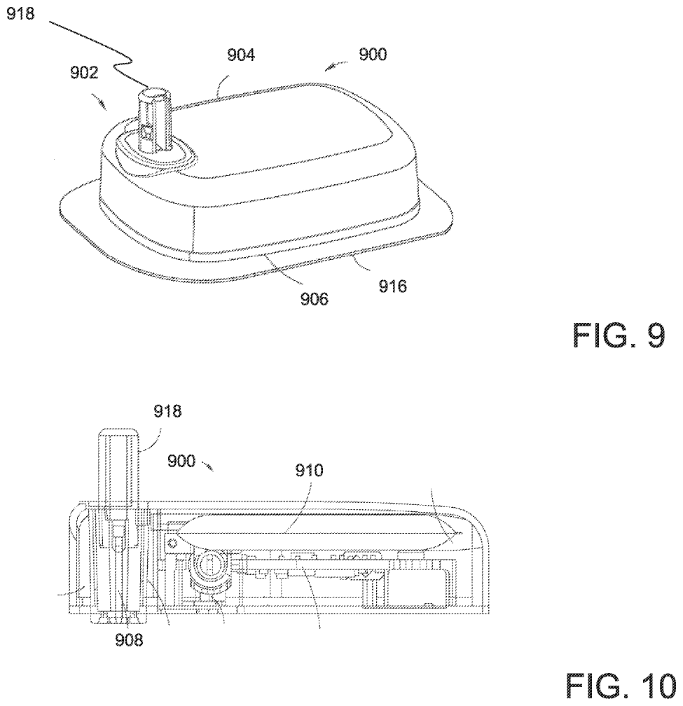

Referring now to FIGS. 9-11, FIGS. 9-11 are illustrations of drug delivery device 900. In some non-limiting embodiments, drug delivery device 900 may be the same or similar to drug delivery device 100, drug delivery device 400, drug delivery device 500, drug delivery device 600, drug delivery device 700, and/or drug delivery device 800. With regard to FIG. 9, FIG. 9 is a perspective view of drug delivery device 900. As shown in FIG. 9, drug delivery device 900 may include drug injection system 902, housing 904, base 906, and button 918. In some non-limiting embodiments, drug delivery device 900 may be a wearable automatic injector. For example, drug delivery device 900 may be an insulin or bone marrow stimulant delivery device. In some non-limiting embodiments, drug delivery device 900 may be applied to (e.g., positioned in contact with) the body of a user. For example, drug delivery device 900 may be mounted onto the skin of a user. In some non-limiting embodiments, based 906 may include adhesive layer 916. In some non-limiting embodiments, button 918 may be configured to allow for manual operation of drug injection system 902 such that a drug may be delivered to the body of the user based on the user depressing button 918.

With regard to FIG. 10, FIG. 10 is a cross-sectional view of drug delivery device 900. As shown in FIG. 10, drug delivery device 900 may include cannula 908, and reservoir 910. As used herein, the term “reservoir” may include any container for injectate, including a bladder, primary container, syringe body, and the like. In some non-limiting embodiments, cannula 908 may include a needle. In some non-limiting embodiments, cannula 908 may include a catheter, which may be a soft, flexible catheter, a rigid catheter, or a combination thereof. In some non-limiting embodiments, cannula 908 may be inserted into the skin of a user. For example, cannula 908 may be inserted into the skin of a user and a needle of cannula may extend into a body of the user. In some non-limiting embodiments, reservoir 910 may include a drug that is to be delivered (e.g., injected) into the body of the user.

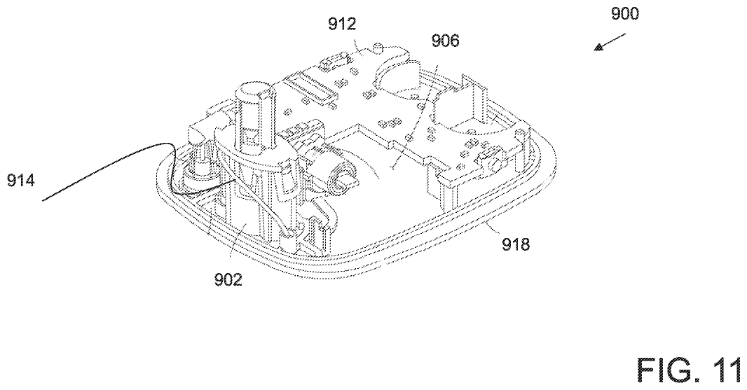

With regard to FIG. 11, FIG. 11 is a perspective view of drug delivery device 900 with housing 904 removed. As shown in FIG. 11, drug delivery device 900 may include drug injection system 902, base 906, printed circuit board 912, and fluid line 914. In some non-limiting embodiments, printed circuit board 912 may include control electronics (e.g., processing device 102) for controlling operation of drug delivery device 900. In some non-limiting embodiments, printed circuit board 912 may include a sensor (e.g., acceleration sensor 404, proximity sensor 504, optical sensor 604, conduction sensor 704, and/or measurement circuit 804 and conductive trace 814), drug injection system 902, and/or a processing device (e.g., processing device 102) attached thereon.

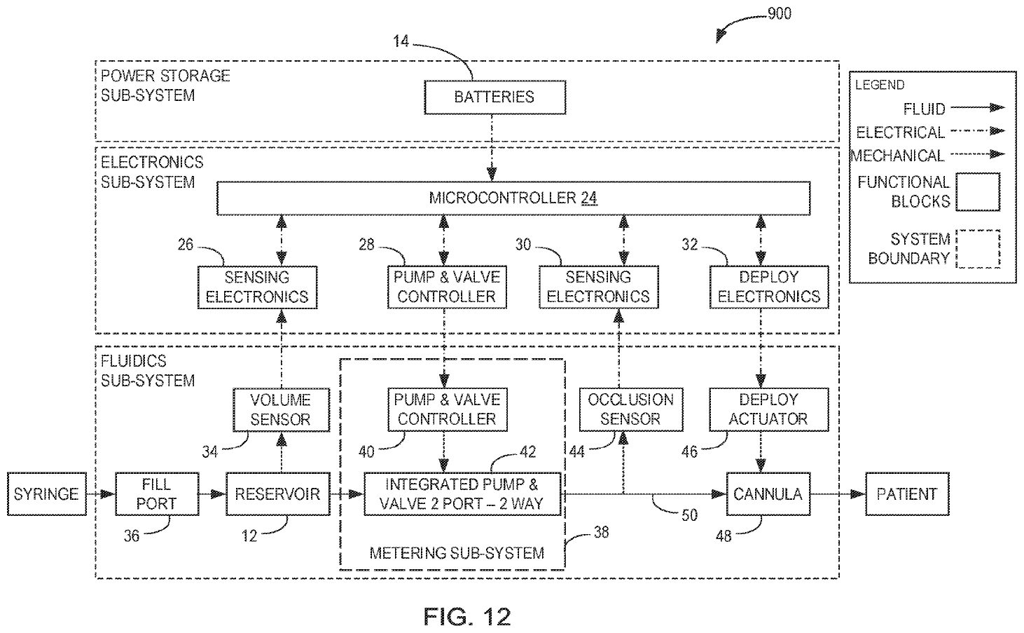

Referring now to FIG. 12, FIG. 12 is a schematic diagram of drug delivery device 900 shown in FIG. 9. As shown in FIG. 12, drug delivery device 900 may include a power storage sub-system, an electronics sub-system, and a fluidics sub-system. In some non-limiting embodiments, power storage sub-system may include power source 14. In some non-limiting embodiments, power source 14 may include a direct current (DC) power source, such as one or more batteries. In some non-limiting embodiments, the electronics sub-system may control the actuation of a drug injection system (e.g., drug injection system 902) of drug delivery device 900. The electronics sub-system may include processing device 24, sensing electronics 26, pump and valve controller 28, sensing electronics 30, and deployments electronics 32. In some non-limiting embodiments, the fluidics sub-system may include reservoir 12, volume sensor 34 for reservoir 12, reservoir fill port 36, and metering sub-system 38, which includes pump and valve actuator 40 and pump and valve mechanism 42. The fluidic sub-system may further include occlusion sensor 44, deploy actuator 46, cannula 48 for insertion into the skin of a user, and fluid line 50 in fluid communication with reservoir 12 and cannula 48. In some non-limiting embodiments, drug injection system 902 may be configured to move cannula 48 from a retracted position positioned entirely within drug delivery device 900 to an extended position where cannula 48 extends outside of drug delivery device 900. In some non-limiting embodiments, cannula 48 is a catheter, which may be a soft, flexible catheter, a rigid catheter, or a combination thereof. In some non-limiting embodiments, drug delivery device 900 may operate in the same manner as discussed in U.S. Pat. No. 10,449,292 to Pizzochero et al., which is incorporated by reference herein in its entirety.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

What is claimed is:

1. A drug delivery device, for delivering a drug to a patient, comprising:
a cannula in fluid communication with a reservoir;
at least one sensor; and
at least one processor in communication with the sensor, wherein the at least one processor is programmed or configured to:
receive data associated with the sensor from the sensor;
determine a characteristic associated with the sensor based on the data received from the sensor, wherein the characteristic is relative movement between the drug delivery device and the patient;
determine whether the characteristic associated with the sensor satisfies a threshold; and
perform an action based on determining that the data received from the sensor satisfies the threshold.

2. The drug delivery device of claim 1, wherein the sensor is an acceleration sensor, wherein, when receiving the data from the sensor, the at least one processor is programmed or configured to:
receive data associated with acceleration of the acceleration sensor from the acceleration sensor;
wherein, when determining the characteristic associated with the sensor, the at least one processor is programmed or configured to:
determine a velocity of the acceleration sensor based on the data associated with acceleration of the acceleration sensor;
wherein, when determining whether the characteristic associated with the sensor satisfies the threshold, the at least one processor is programmed or configured to:
determine whether the velocity of the acceleration sensor satisfies a threshold value of velocity; and
wherein, when performing the action, the at least one processor is programmed or configured to:
perform the action based on determining that the velocity of the acceleration sensor satisfies the threshold value of velocity.

3. The drug delivery device of claim 1, wherein the sensor is an acceleration sensor, wherein, when receiving the data from the sensor, the at least one processor is programmed or configured to:
receive data associated with acceleration of the acceleration sensor from the acceleration sensor;
wherein, when determining the characteristic associated with the sensor, the at least one processor is programmed or configured to:
determine an amount of inertial force of acceleration sensor based on the data associated with acceleration of the acceleration sensor;
wherein, when determining whether the characteristic associated with the sensor satisfies the threshold, the at least one processor is programmed or configured to:
determine whether the amount of inertial force of the acceleration sensor satisfies a threshold value of force; and
wherein, when performing the action, the at least one processor is programmed or configured to:
perform the action based on determining that the inertial force of the acceleration sensor satisfies the threshold value of force.

4. The drug delivery device of claim 3, wherein, when determining the amount of inertial force, the at least one processor is programmed or configured to:
determine a first amount of inertial force in a first direction based on first data associated with acceleration of the acceleration sensor in a first direction;
determine a second amount of inertial force in a second direction based on second data associated with acceleration of the acceleration sensor in a second direction;
wherein, when determining whether the inertial force of the acceleration sensor satisfies the threshold value of force, the at least one processor is programmed or configured to:
determine whether the first amount of inertial force in the first direction satisfies a first threshold value of force;
determine whether the second amount of inertial force in the second direction satisfies a second threshold value of force; and
wherein, when performing the action, the at least one processor is programmed or configured to:
perform the action based on determining that the first amount of inertial force in the first direction satisfies the first threshold value of force or that the second amount of inertial force in the second direction satisfies the second threshold value of force.

5. The drug delivery device of claim 1, wherein the sensor is an acceleration sensor, wherein, when receiving the data from the sensor, the at least one processor is programmed or configured to:
receive data associated with acceleration of the acceleration sensor from the acceleration sensor;
wherein, when determining the characteristic associated with the sensor, the at least one processor is programmed or configured to:
determine a first vector value of acceleration for the acceleration sensor in a first direction based on the data associated with acceleration of the acceleration sensor;
determine a second vector value of acceleration for the acceleration sensor in a second direction based on the data associated with acceleration of the acceleration sensor; and
determine a resultant acceleration vector of acceleration for the acceleration sensor based on the first vector value of acceleration of the acceleration sensor in the first direction and the second vector value of acceleration of the acceleration sensor in the second direction;
wherein, when determining whether the characteristic associated with the sensor satisfies the threshold, the at least one processor is programmed or configured to:
determine whether the resultant acceleration vector of acceleration for the acceleration sensor satisfies a threshold value of acceleration; and
wherein, when performing the action, the at least one processor is programmed or configured to:
perform the action based on determining that the resultant acceleration vector of acceleration for the acceleration sensor satisfies the threshold value of acceleration.

6. The drug delivery device of claim 1, wherein the sensor is an acceleration sensor, wherein the at least one processor is further programmed or configured to:
determine an orientation of the acceleration sensor; and
wherein, when determining the characteristic associated with the sensor, the at least one processor is programmed or configured to:
determine the characteristic associated with the acceleration sensor based on the orientation of the acceleration sensor.

7. The drug delivery device of claim 1, wherein the at least one processor and the sensor are positioned on a printed circuit board (PCB).

8. The drug delivery device of claim 1, further comprising:
a housing; and
a drug injection system;
wherein the at least one processor and the drug injection system are positioned within the housing.

9. The drug delivery device of claim 8, wherein the drug injection system comprises a cannula configured to be inserted into at least one of a vascular, a dermis, an intradermal tissue, a subcutaneous tissue and a muscle tissue of a user.

10. The drug delivery device of claim 1, wherein the sensor is a contact sensor, wherein, when receiving the data from the sensor, the at least one processor is programmed or configured to:
receive data associated with a position of the contact sensor from the contact sensor;
wherein, when determining the characteristic associated with the sensor, the at least one processor is programmed or configured to:
determine a condition of the contact sensor with regard to the contact sensor being adjacent a body of a user based on the data associated with a position of the contact sensor;
wherein, when determining whether the characteristic associated with the sensor satisfies the threshold, the at least one processor is programmed or configured to:
determine whether the condition of the contact sensor satisfies a threshold value associated with the contact sensor being adjacent the body of the user; and
wherein, when performing the action, the at least one processor is programmed or configured to:
perform the action based on determining that the condition of the contact sensor satisfies the threshold value associated with the contact sensor being adjacent the body of the user. 11 The drug delivery device of claim 1, wherein the threshold comprises a threshold value based on a working mode of the drug delivery device.

12. A drug delivery device comprising:
a housing;
a drug injection system;
a sensor; and
at least one processor in communication with the sensor;
wherein the at least one processor, the sensor, and the drug injection system are positioned within the housing; and
wherein the at least one processor is programmed or configured to:
receive data associated with the sensor from the sensor;
determine a characteristic associated with the sensor based on the data received from the sensor;
determine whether the characteristic associated with the sensor satisfies a threshold; and
perform an action based on determining that the data received from the sensor satisfies the threshold.

13. The drug delivery device of claim 12, wherein the sensor is an acceleration sensor, wherein, when receiving the data from the sensor, the at least one processor is programmed or configured to:
receive data associated with acceleration of the acceleration sensor from the acceleration sensor;
wherein, when determining the characteristic associated with the sensor, the at least one processor is programmed or configured to:
determine a velocity of the acceleration sensor based on the data associated with acceleration of the acceleration sensor;
wherein, when determining whether the characteristic associated with the sensor satisfies the threshold, the at least one processor is programmed or configured to:
determine whether the velocity of the acceleration sensor satisfies a threshold value of velocity; and
wherein, when performing the action, the at least one processor is programmed or configured to:
perform the action based on determining that the velocity of the acceleration sensor satisfies the threshold value of velocity.

14. The drug delivery device of claim 12, wherein the sensor is an acceleration sensor, wherein, when receiving the data from the sensor, the at least one processor is programmed or configured to:
receive data associated with acceleration of the acceleration sensor from the acceleration sensor;
wherein, when determining the characteristic associated with the sensor, the at least one processor is programmed or configured to:
determine an amount of inertial force of the acceleration sensor based on the data associated with acceleration of the acceleration sensor;
wherein, when determining whether the characteristic associated with the sensor satisfies the threshold, the at least one processor is programmed or configured to:
determine whether the amount inertial force of the acceleration sensor satisfies a threshold value of force; and
wherein, when performing the action, the at least one processor is programmed or configured to:
perform the action based on determining that the inertial force of the acceleration sensor satisfies the threshold value of force.

15. The drug delivery device of claim 12, wherein the sensor is an acceleration sensor, wherein, when receiving the data from the sensor, the at least one processor is programmed or configured to:
receive data associated with acceleration of the acceleration sensor from the acceleration sensor;
wherein, when determining the characteristic associated with the sensor, the at least one processor is programmed or configured to:
determine a first vector value of acceleration for the acceleration sensor in a first direction based on the data associated with acceleration of the acceleration sensor;
determine a second vector value of acceleration for the acceleration sensor in a second direction based on the data associated with acceleration of the acceleration sensor; and
determine a resultant acceleration vector of acceleration for the acceleration sensor based on the first vector value of acceleration of the acceleration sensor in the first direction and the second vector value of acceleration of the acceleration sensor in the second direction;
wherein, when determining whether the characteristic associated with the sensor satisfies the threshold, the at least one processor is programmed or configured to:
determine whether the resultant acceleration vector of acceleration for the acceleration sensor satisfies a threshold value of acceleration; and
wherein, when performing the action, the at least one processor is programmed or configured to:
perform the action based on determining that the resultant acceleration vector of acceleration for the acceleration sensor satisfies the threshold value of acceleration.

16. The drug delivery device of claim 12, wherein the sensor is a contact sensor, wherein, when receiving the data from the sensor, the at least one processor is programmed or configured to:
receive data associated with a position of the contact sensor from the contact sensor;
wherein, when determining the characteristic associated with the sensor, the at least one processor is programmed or configured to:
determine a condition of the contact sensor with regard to the contact sensor being adjacent a body of a user based on the data associated with a position of the contact sensor;
wherein, when determining whether the characteristic associated with the sensor satisfies the threshold, the at least one processor is programmed or configured to:
determine whether the condition of the contact sensor satisfies a threshold value associated with the contact sensor being adjacent the body of the user; and
wherein, when performing the action, the at least one processor is programmed or configured to:
perform the action based on determining that the condition of the contact sensor satisfies the threshold value associated with the contact sensor being adjacent the body of the user.

17. The drug delivery device of claim 16, wherein the contact sensor comprises at least one of:
a proximity sensor;
an optical sensor;
a conduction sensor; or
a measurement circuit.

18. The drug delivery device of claim 17, wherein the proximity sensor is at least one of a capacitive sensor and an ultrasonic sensor.

19. A drug delivery device comprising:
at least one processor programmed or configured to:
receive data associated with a sensor from the sensor;
determine a characteristic associated with the sensor based on the data received from the sensor;
determine whether the characteristic associated with the sensor satisfies a threshold; and
perform an action based on determining that the data received from the sensor satisfies the threshold;
wherein, when performing the action, the at least one processor is programmed or configured to perform at least one of:
provide an alarm;
provide a notification;
cease delivery of a drug from the drug delivery device;
store data associated with an event;
communicate data associated with the event to a remote device; or
any combination thereof.

20. The drug delivery device of claim 18, further comprising:
a housing;
a drug injection system;
an adhesive layer;
wherein the at least one processor and the drug injection system are positioned within the housing; and
wherein the adhesive layer is configured to be positioned on a body of a user.

21. The drug delivery device of claim 19, wherein the drug injection system comprises a cannula configured to be inserted into at least one of a vascular, a dermis, an intradermal tissue, a subcutaneous tissue and a muscle tissue of a patient.
